(19) United States
(12) Patent Application Publication
(10) Pub. No.: US 2011/0071490 A1
(43) Pub. Date: Mar. 24, 2011

(54) SYSTEM AND PROCEDURE FOR PLACING A MEDICAL DEVICE PROXIMATE AN OSTIAL LESION USING A CATHETER ASSEMBLY

(75) Inventors: Elias Habib Kassab, Grosse Pointe Shores, MI (US); Mark Edwin Zyzelewski, Kalamazoo, MI (US)

(73) Assignee: KASSAB INTERVENTIONAL DEVICES, LLC ("KIDS"), Dearborn, MI (US)

(21) Appl. No.: 12/562,713

(22) Filed: Sep. 18, 2009

(51) Int. Cl. A61M 31/00; A61M 25/00; A61M 39/10

(52) U.S. Cl. 604/500; 604/264; 604/533

(57) ABSTRACT

A catheter system and method 10 for placing a medical device proximate an ostial lesion. The catheter system 10 has a guide catheter 14 with a proximal end 16 and a distal end 18. A sheath 22 with an internal lumen 24 surrounds the guide catheter 14. The sheath 22 has a near end 26 and a far end 28. A cavity 32 is defined between the outside of the guide catheter 14 and the inside of the sheath 22. One or more longtitudinal connecting components 34 extend axially within the cavity with an actuating mechanism 38. An ostial locating member 42 extends from the far end 22 of the sheath so that the ostial locating member 42 lies outside the guide catheter 14. The ostial locating member 42 has at least two flexible, radially extending struts 44 that terminate with radio-opaque feet 46 that lie in an ostial plane 48 when they are advanced in a distal direction from outside the distal end 40 of the guide catheter 14 and against the interior wall of the aorta 50. The ostial locating member 42 permits placement of the radio-opaque feet 46 proximate to or in the ostial plane 48 when the ostial locating member 42 is advanced distally after the expandable distal legs 70 have been expanded radially outward beyond the distal end 18 of the guiding catheter 14.

### BACKGROUND OF THE INVENTION

1. Field of the Invention

The invention relates to a catheter assembly for positioning a medical device, such as a stent, at or near the site of an ostial lesion.

2. Background of the Invention

In medicine, a “stent” is a man-made tube that is inserted into a natural passage/conduit in the body to prevent, or counteract a disease-induced, localized flow constriction. http://en.wikipedia/org/wiki/stent#peripheralvascular. As used herein, the term “stent” includes for example an endovascular, cylindrical, mesh-like but dilatible structure which is inserted into various atherosclerotic arteries to maintain the patency of the vessel. Usually the diseased artery has been dilated to remove the blockage with an angioplasty balloon, which has been introduced percutaneously within a tube or catheter inserted into the right femoral artery, for instance, and guided to the diseased arterial site. Conventionally, after the stent is inserted, it is left in place to maintain the opening in the previously blocked artery. Predictable, repeatable and precise placement of the stent may be challenging using conventional techniques.

During interventional vascular procedures, situations arise where a stent must be placed at the ostium of an artery. The ostium is the opening (or mouth) of an artery into another artery, such as the aorta. Ostial lesions involve for example the junction of the aorta and the origin of the right coronary artery, left main stem or a saphenous vein graft. Such lesions are called “aorto-ostial”. Other ostial lesions occur at the origin of the left anterior descending artery of the left circumflex artery arising from the bifurcation of the distal left main stem. Still other lesions may recur in the iliac and/or femoropopliteal arteries. As used herein, the term “ostial lesions” is used to describe these types of lesions. Inaccurate placement of the stent at the ostium of an artery can result in multiple risks to the patient, repeated procedures and related costs.

The location of ostial lesions makes them difficult to treat by percutaneous coronary intervention. Accurate stent positioning to avoid stent protrusion into the left main stem, for example, remains challenging. Aziz & Ramsdale, “SIROLIMUS—ELUTING STENTS FOR THE TREATMENT OF ATHEROSCLEROTIC OSTIAL LESIONS”, 17 Jnl. Invasive Cardiology, Jan. 3, 2005. If the stent is deployed too proximal to the ostium, the stent could protrude into the aorta. In such cases, future re-engagement of the vessel could be difficult. The only option for a patient who has restenosis of an artery could be bypass surgery. If the stent is deployed too distal to the ostium, additional stents may be required to fully open the lesion. This will result in increased cost of the procedure, and a higher risk for thrombosis and restenosis of the stented area.

There are devices that aid in the placement of stents at the ostium of vessels. One such device is the “Ostial Pro™” from Ostial Solutions, LLC. The Ostial Pro is a nitinol device that is positioned within a guiding catheter. http://www3.interscience.wiley.com/journal/117916410/abstract. It has distal, self-expanding legs that are advanced just ahead of the tip of the guiding catheter after the ostial lesion has been crossed by a coronary guidewire and stent delivery system. Id. The expanded nitinol legs prevent the entry of the guiding catheter into the target vessel, mark the plane of the aortic wall, and align the tip of the guide with the aorto-ostial plane. Id. Such devices include products which travel within a guide catheter to aid in locating the ostium and thus occupy a portion of the limited internal diameter of such catheters.

To locate the true ostium of an artery, a suitable positioning device must create a visual or tactile feedback system that can be utilized during an interventional procedure. Visual and tactile indicators come in many forms. A visual indicator utilizes radio-opaque materials that can be seen under fluoroscopy. These indicators create a reference point for locating the ostial interface. A tactile indicator utilizes a feature that transfers a tactile feel to the proximal end of the device to inform the surgeon, who may be located one meter away from the lesion, that the device is in the proper position. But accurate tactile feedback requires unimpeded, smooth axial movement of such features along and within the length of a catheter that may have a small internal diameter.

The typical guide catheter for delivering, retrieving or re-positioning stents is made of a reinforced tubular polymer. The guide catheter comes in various French sizes. Typically, these sizes range between 6, 7, and 8 French. The French catheter scale is commonly used to measure the outer diameter of cylindrical catheters. http://en.wikipedia.org/wiki/Frenchcatheterscale. The diameter in millimeters of the catheter equals the French size divided by 3. For example, if the French size is 9, the diameter is 3 mm. Id.

As used herein, the term “catheter” means a tube that can be inserted into a body cavity, duct or vessel. http://en.wikipedia.org/wiki/catheter. Catheters allow drainage, injection of fluids or access by surgical devices or instruments. The process of inserting a catheter is called “catheterization”. In most uses, a catheter is a thin, flexible tube. But in some uses, it may be a larger solid tube, for example, to facilitate placement into a particular part of the body. Various catheter tips or guide wires can be used to guide the catheter into the target vessel.

A guide catheter is a conduit for introducing interventional devices during an endovascular procedure. The proximal (extracorporeal) end of the guide catheter may incorporate a luer lock. Conventionally, a luer lock is a system of small-scale fluid fittings that is used for making leak-free connections between a male-taper fitting and its mating female part on medical instruments. http://en.wikipedia.org/wiki/LuerTaper. Luer lock fittings are securely joined by a tabbed hub on the female fitting which screws into threads in a sleeve on the male fitting. Id. Luer components are manufactured from metal or plastic and are available from many companies worldwide. Id. “Luer-LOK” is a registered trademark of Becton Dickinson. In the literature and in this patent application, a “Luer-Lok” style connector is generically referred to as a “luer-lock connector”.

Thus, the luer lock is the conduit that allows a guide catheter to interface with external devices to introduce wires, balloons, stents, contrast, and/or prevent the back flow of blood out of the guide catheter. The distal (intracorporeal) end of the guide catheter is typically formed into a shape to help the introduction of devices into the vasculature. The shape is formed during the manufacturing of the catheter. Differently shaped catheters are used in different vascular applications.

During a typical procedure, the guide catheter is engaged into the artery to be stented. When engaged, the distal end of the guide catheter is located within the ostial interface. Due to the proximity of the distal end of guide catheter to the ostial interface, it would be desirable to use the distal end of the guide catheter itself to position an ostial locator device.

Illustrative of the prior art are U.S. Pat. Nos. 5,749,890; 6,458,151; and 6,659,981; and U.S. Publication Nos. 2007/0225790, 2008/0082155 and 2008/0228146.

One consequence of having an ostial locating device inside the catheter, as described in U.S. Publication Nos. 2007/0225790 and 2008/0082155 is that the effective inside diameter of the catheter is reduced. As a consequence, less space is available without interference for use by other devices, such as stents.

For example, a typical inside diameter of a catheter may be 1/80,000 inches. A locating device having a wall thickness of 1/5,000 inches may be used, which when doubled leaves an effective amount of useful space of 1/70,000 inches. This may make it difficult to manipulate uninterruptedly and without interference a stent that may be suitable for use in a patient's legs, which may other things being equal, require a larger stent. Simply stated, the stent needs room to maneuver within the internal diameter of the catheter and locating device. Such clearance issues affect the feel that is transmitted to the physician.

One adverse consequence of such arrangements is that a stent may become damaged. In some cases, the stent may be fragile and easily bent by unwanted interference with a surrounding structure. If bent, fracture may result. Stent fracture may not be apparent during an operational procedure. Indeed, it may not be evident until some time later when the stent exhibits fatigue fracture upon repeated exposure to blood flow that may pulsate, for example, every two seconds. Another adverse consequence is that a ruptured stent may pierce a balloon that is used in angioplasty, or cause the balloon to inflate non-uniformly.

In light of such observations, it would be desirable to have an ostial locating device that is located on the outside of the catheter so that there is less risk of interference between the inside lumen of the catheter and a medical device such as a stent that passes therethrough.

### SUMMARY OF THE INVENTION

The inventive catheter system solves ostial-aorta positioning problems by creating an assembly that precisely and predictably locates the ostium of an artery using a guide catheter. The invention offers design variations of the guide catheters currently on the market. In one embodiment, the assembly incorporates a locating member that can be deployed on and when actuated lies preferably ahead of the distal outside diameter of the guide catheter, thereby liberating scarce space within the catheter. The inventive locating member is actuated by a mechanism which interfaces with the surgeon at the proximal end of the catheter near a luer lock.

In several embodiments, (1) the luer lock is incorporated into a proximal assembly, and (2) the guide catheter lies within a thin walled sheath that is associated with the locating members. Preferably, a typical guide catheter reinforced tube is used. The cavity created between the guide catheter (inner tube) and the sheath (outer tube) creates an annular path that houses one or more connecting components that are positioned between the proximal actuation and the distal ostial locating member.

In each embodiment, actuation at the proximal end of the remotely situated locating member is performed by a sliding mechanism that is provided extracorporeally, proximal to the surgeon. This mechanism may take many forms, such as helical or rotational forms, but for simplification, a linear mechanism is shown in all embodiments.

One recommended procedure for placing a medical device such as a stent proximate ostial lesion involves positioning the locating device on an outside surface of the guiding catheter. The locating device in one embodiment has flexible distal, radially and outwardly extending struts that are advanced to a deployed position that is just ahead of the distal end of the guiding catheter after the ostial lesion has been crossed. After the struts have been deployed, they prevent further incursion of the guiding catheter into the artery into which the stent is to be placed. When deployed, the struts define the plane of the aortic wall. The catheter tip may then be advanced so that it lies in or near the ostial plane. In that position, the struts help align the distal end of the guiding catheter with the ostial plane. A stent or other device can then be predictably and accurately placed in the ostial lesion by a controlled emergence from the catheter tip, unimpeded by the locating member.

### BRIEF DESCRIPTION OF THE DRAWINGS

FIG. 1 is an overall perspective view of several aspects of the inventive catheter assembly;

FIG. 1A is an enlargement of a distal portion of the embodiment depicted in FIG. 1, illustrating an -ostial locating member (a cage in which the “bars” are links or strips) in a collapsed position on the outside of a catheter;

FIG. 2 illustrates a guide catheter extending from a proximal portion of the inventive catheter system;

FIG. 2A is an enlarged perspective view of a cross-section of a distal portion of the guide catheter assembly illustrating an outer sheath, an annular actuating cavity that houses an actuating compare end, and an inner (catheter) tube;

FIG. 3 is a side view of the inventive system illustrating a linear actuator at a proximal portion moved to an open (locator-actuating) position;

FIG. 3A is an enlargement of a side view, distal portion of the inventive system in which the actuator and locating members are deployed in an open position;

FIG. 3B is a perspective view of a distal portion of the inventive system, in which the distal actuator and locating member are deployed in an open configuration;

FIG. 4 illustrates a second embodiment of the inventive system in which the linear actuator is locked so that the locating member (in which the “bars” are wires) lies in a collapsed position;

FIG. 4A is an enlargement of that portion of FIG. 4 which illustrates the locating member in a collapsed position, substantially flush with the outside of the sheath (outer tube);

FIG. 5 illustrates a third (preferred) embodiment in which the linear actuator is moving toward an open position and correspondingly the cage-shaped ostial locating member located at the distal end lies in an open position;

FIG. 5A is an enlargement of the distal portion of FIG. 5, showing the locating member in a deployed position;

FIG. 5B is a perspective view of the embodiment depicted in FIG. 5A;

FIG. 5C is a side elevation view of that shown in FIG. 5B;

FIG. 6 depicts a fourth embodiment, in which the linear actuator is locked in a collapsed position and distal legs in the form of struts likewise repose in a collapsed position;

FIG. 6A is an enlargement of the distal portion of FIG. 6;

FIG. 7 depicts a fifth embodiment, in which the linear actuator is moved to an open position and the distal legs likewise lie in an open position;

FIG. 7A is an enlargement of a distal portion of FIG. 7 illustrating their extension outside the catheter, but from the inside of the sheath (outer tube);

FIG. 7B is a perspective view of the embodiment depicted in FIG. 7A;

FIG. 8 is a perspective view of a sixth embodiment, showing curved legs in a deployed configuration;

FIG. 9 is a side view of the embodiment shown in FIG. 8;

FIG. 10 is an end view thereof;

FIG. 11 is a cross-sectional view a of catheter that includes in its wall a wire actuating component according to the present invention;

FIG. 12 is a cross-sectional view of an alternative embodiment to that depicted in FIG. 11; and

FIG. 13 depicts a center tube (catheter) that has a lumen for introducing interventional devices; an outer tube (sheath); and a crescent-shaped actuating cavity that lies between the tubes within which is located an actuating component that may move axially.

### DETAILED DESCRIPTION OF THE PREFERRED EMBODIMENT(S)

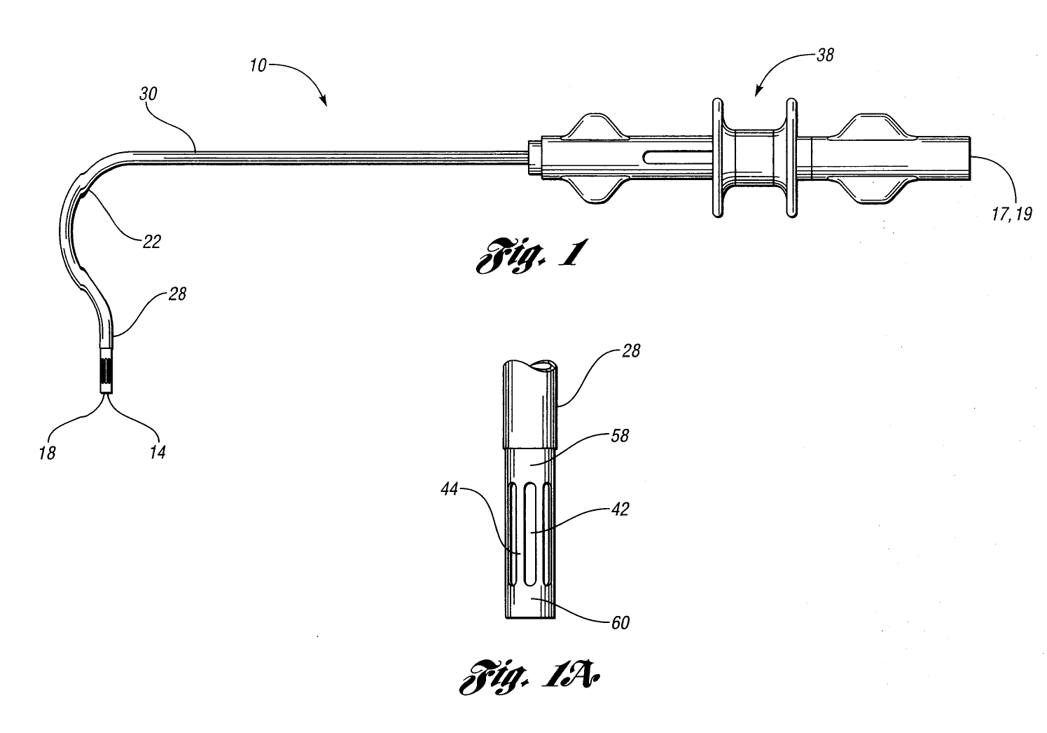

Turning first to FIG. 1 of the drawings, there is depicted a catheter system 10 including a guide catheter 14 that has a proximal end 16 which interfaces via an actuating mechanism 38 with a surgeon and external devices 17 that are connected through a luer-lock 19 or comparable arrangement. The luer-lock 19 is attached to tubing associated with the guide catheter 14. Conventionally, the luer-lock 19 is incorporated within a proximal hub (not shown) which is a stationary member. The proximal hub is a suitable area for incorporating actuation components that interface with an ostial locating member.

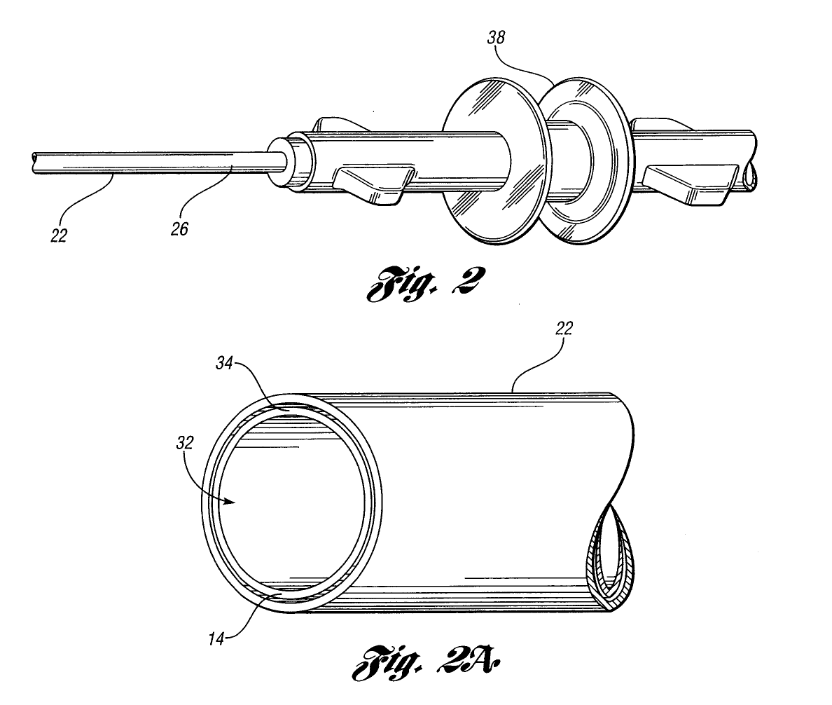

Further distally, guide catheter 14 in one embodiment can be considered as a reinforced tubing with a single lumen 52 (FIGS. 2-2A). Typically, the tubing 14 has an external hydrophillic coating to improve lubricity and is available in a variety of lengths.

Returning to FIG. 1, it will be appreciated that the guide catheter 14 may be provided with variously shaped distal ends 28 that support an ostial locating member 42. To accommodate this member, the catheter 14 is formed during manufacturing in multiple shapes, and one is depicted in FIG. 1 as a non-limiting example. Such shapes (such as one arm of a pair of eyeglasses) improve the surgeon's ability to access various areas in the vasculature. During an endovascular procedure, the shape is preferably maintained to ensure arterial engagement.

Continuing with primary reference to FIG. 1, the distal end 18 of the guide catheter 14 interfaces with arteries. Typically, it is soft and non-traumatic to prevent arterial damage.

In the description of various alternative inventive embodiments, it will be appreciated that the luer-lock 19 or comparable subsystem will preferably be incorporated into proximal assembly associated with the linear actuator 38. A feature that is also common to the alternate inventive embodiments is the guide catheter 14, including a covering sheath 22 or tubing. In the catheter system described and depicted, the sheath 22 preferably has a thin wall. In one embodiment, an actuation cavity 32 is created between the guide catheter (inner tube) 14 and the sheath (outer tube) 22. That cavity 32, which may be annular or crescent-shaped, houses for example ribbon-like, arcuate connecting or actuating components 34 between the proximal actuating mechanism 38 and distal ostial locating member 42. (FIG. 2A) In FIG. 2A, the connecting component 34 is depicted as a thin ribbon-like structure that has an arcuate cross-section. Thus, it can readily slide within the cavity 32 formed between the inner catheter 14 and the outer sheath 22.

Actuation of the alternative ostial locating members 42 is enabled by the linear or axial sliding of one or more connecting components 34. Optionally, actuation could be achieved by other means for actuation, such as those to be described below.

Various alternative embodiments of the inventive ostial locating member 42 of the catheter system 10 will now be discussed.

### Embodiment 1

### Distal Cage with Linear Slide Actuation (FIGS. 1-3B)

In the embodiment depicted in FIGS. 1-3B, a distal cage 56 extends from the outside diameter of the far end 28 of the sheath 22 (FIG. 1A). The basal end 58 end of the distal cage 56 is fixedly attached to or extends from the far end 28 of the sheath 22. The basal end 58 of the distal cage 56 is fixedly attached to one or more wires or ribbons 34 (or similar actuating components) that have longitudinal or axial stiffness so they may communicate longitudinally directed forces associated with action or reaction (push or push-back) which extend to the proximally located linear actuator 38 (FIGS. 2 & 13). The apex end 60 of the distal cage 56 can slide along the outside of the guide catheter tubing 14.

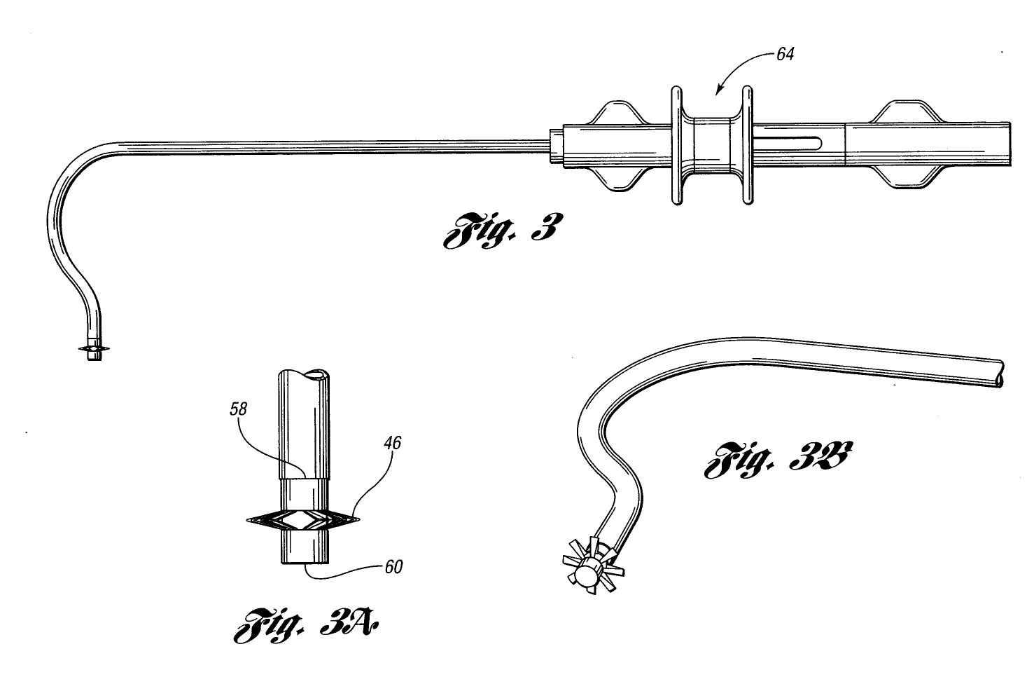

When the linear actuator 38 is moved to the open position 64 (FIG. 3), the distal cage 56 expands to form a disk-like shape (FIGS. 3A, 3B). Linear motion of the actuating mechanism 38 is transferred via a connecting component to the sliding portion 60 of the distal cage 56. When the apex end 60 of the distal cage 56 slides nearwardly, the connecting component 34 moves axially in the cavity 32 and the disk form of the ostial locating member 42 is created.

Preferably, the actuating mechanism 38 is biased to the distal position so that upon release, the cage 56 opens. Accordingly, the material for the distal cage 56 is preferably a shape memory material (e.g., nitinol).

As contemplated herein, the disc-shaped distal end of the guide catheter preferably is made from a shape memory alloy that can be deformed and then instantly revert to its original shape when the stress is removed. This is the result of pseudo-elasticity. Accordingly, the disc-shaped distal end can be bent, twisted and pulled before reforming its shape when released. Thus, they have the attribute of longevity because it appears that almost no amount of bending will result in permanent plastic deformation. As used herein, the term “shape memory material” include alloys or polymers that remember their shape to which they return after being deformed, often by applying heat to the material. http://en.wikipedia.org/wiki/Shapememoryalloy. The three main types of shape memory alloy are the cooper-zinc-aluminum-nickel, copper-aluminum-nickel, and nickel-titanium (NiTi) alloys. NiTi alloys are generally more expensive. One of their medical uses involves using pseudo-elastic properties of the alloy. Id. If desired, all or part (e.g., the tips) of the distal portions of the catheter assembly 10 include a radio opaque alloy to facilitate fluoroscopic visualization. When the distal cage 56 is in the open position, it can lie flush with the beginning of the ostium of the vessel, and be used to locate its proximal portion—the ostial interface.

In operation, the same catheter assembly 10 that delivers a stent has a distal end 42 that can be located adjacent to the ostium by engagement of the locating member 42 where the opening lies.

### Embodiments 2 & 3

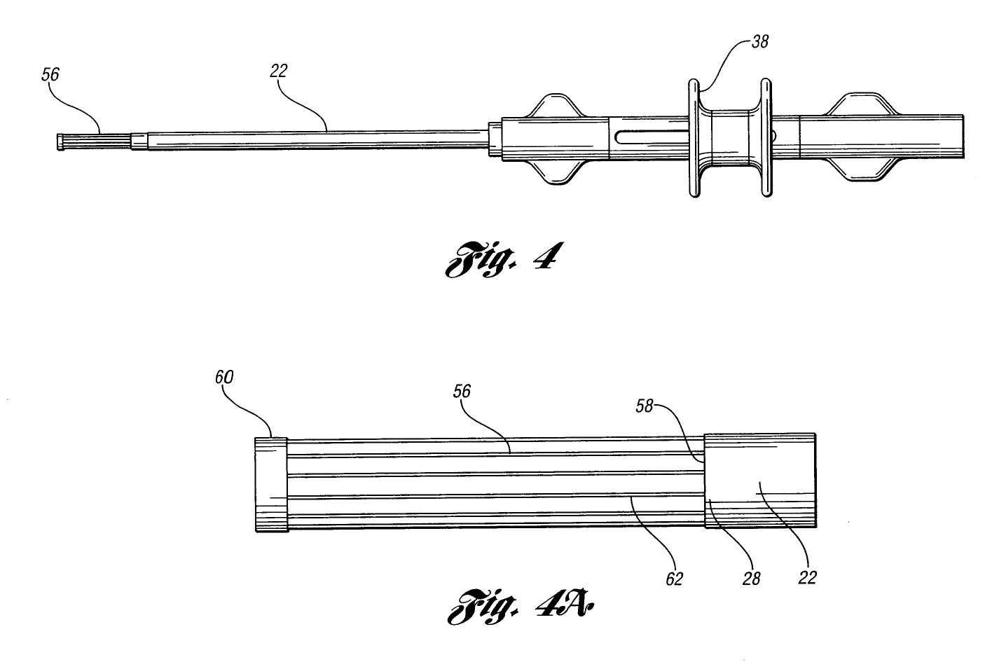

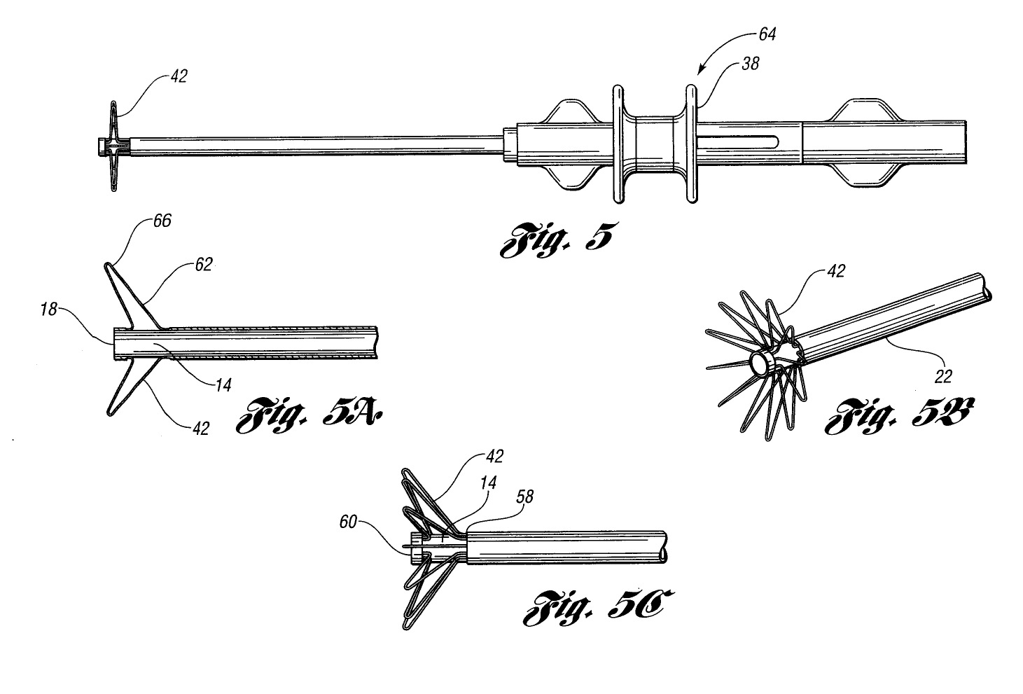

### Distal Cage with Linear Slide Actuation (FIGS. 4-5B)

In embodiment 2, a mesh of wires forms a cage 56 that serves as a distal locating member 42.

The distal cage 56 is attached to the outside diameter of the distal end of the sheath 22. The base 58 of the distal cage 56 is fixedly attached to the far end 28 of the sheath 22. The base 58 of the cage 56 is fixedly attached to a wire, ribbon, or similar connecting component 34 which extends rearwardly to the linear actuator 38 (FIG. 4). The apex end 60 of the cage 56 can move along the outside diameter of the guide catheter tubing 14.

When the linear actuator is moved to the open position, the cage 56 expands to form a disk-like shape. A preferred embodiment is shown in FIGS. 5, 5A, 5B & 5C. Linear motion of the actuator 38 is transferred to a sliding portion of the proximal (base) end 58 of the cage 56. When the base end 58 of the cage 56 slides forwardly, the locating member 42 is formed. The material for the cage 56 is preferably a shape memory material (e.g., nitinol).

When the links 62 of the cage 56 is in the open position, the links 62 preferably lie ahead of the distal end 18 of the catheter 14. In that placement, the links 62 can interface with ostium of the vessel, thus locating the ostial interface predictably, speedily and precisely before emplacing a balloon on stent.

The embodiment of the cage 56 shown in FIGS. 5A & 5B has multiple links or wires 62 in a linear or axial orientation (FIG. 4A). Optionally, the links 62 could be formed in a weaved configuration (e.g. a mess) to make the locating member 42 more robust when formed into the open/disk shape.

Upon inspection of FIGS. 5, 5A, 5B and 5C, it will be appreciated that the locating member 42 assumes a concave configuration (not unlike a toilet plunger) that effectively serves as a cup that can be placed adjacent an ostium wall. When expanded, the locating member 42 or mesh extends past the distal end 18 of the catheter 14, as best shown in FIGS. 5A and 5C. This allows the locating member 42 to interface with a vessel wall without the distal end of the catheter extending into the target vessel. Otherwise, if the distal end 18 of the catheter 14 were to extend into the target vessel, the penetrating distal end 18 may interfere with expansion of an angioplasty balloon.

It will be appreciated that the embodiment depicted in FIG. 5 illustrates a distal cage 56 with 12 outwardly extending links or tentacles 62. But the invention is not so limited. Fewer or more radially extending tentacles 62 can be deployed. Similarly with FIG. 3B.

### Embodiments 4 & 5

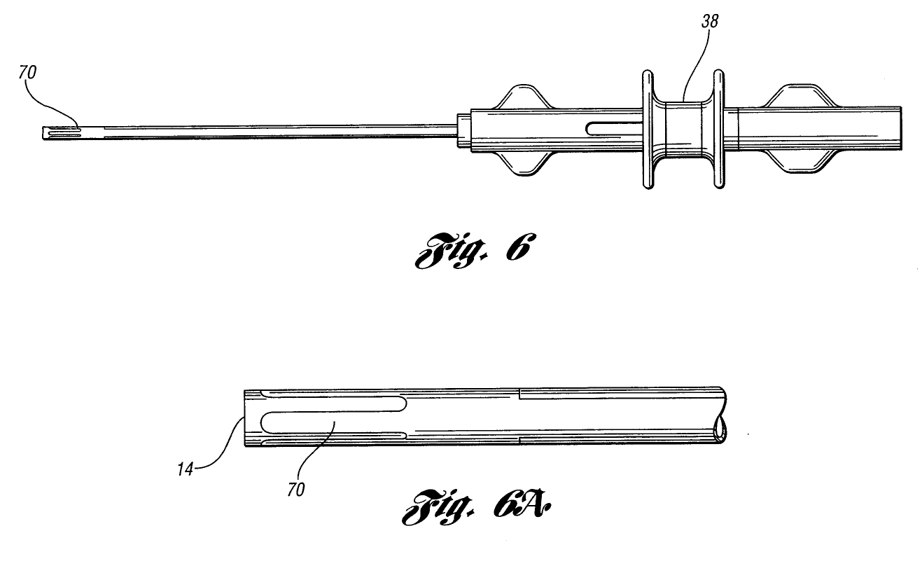

### Distal Legs with Linear Slide Actuation (FIGS. 6-7B)

In a fourth embodiment (FIG. 6A), distal legs are formed from the sheath tubing 22 to create a multi-pronged locating member 42. Although other techniques could be used, one way to prepare the distal legs 70 is by a laser cutting operation.

The distal legs 70 are slid onto the catheter 14, but are not fixedly attached to the outside diameter of the catheter 14. The distal legs 70 can move relative to the catheter 14. they lie outside the catheter 14, but inside the sheat 22.

The proximal end of the distal legs 70 is fixedly attached to a connecting component 34 which extends back to the linear actuator 38. The entire distal leg component 70 can move along the outside diameter of the guide catheter 14 tubing within the actuation cavity 32.

When the linear actuator 38 is moved to the open position, the distal legs 70 slide axially within the actuation cavity 32. When moved to the completely open position, the distal legs 70 extend beyond the distal end 18 of the catheter 14. When extended, the multi-pronged structure forms a landing surface (FIG. 7B). The material for the distal legs 70 is preferably a shape memory material (e.g., nitinol).

When the distal legs 70 are in the open position, they can interface with ostium of the vessel, and be used to locate the ostial interface.

### Embodiment 6

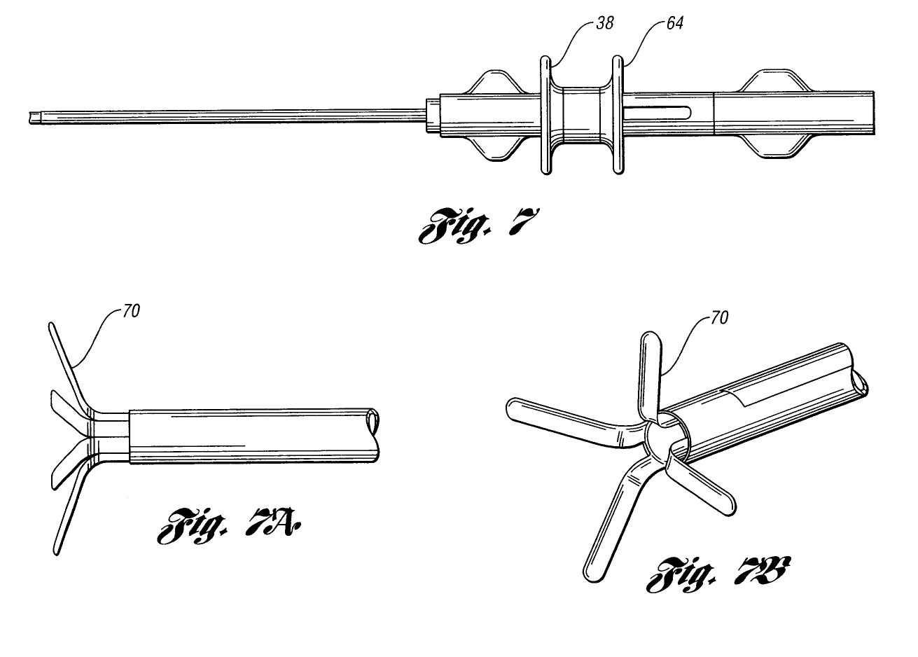

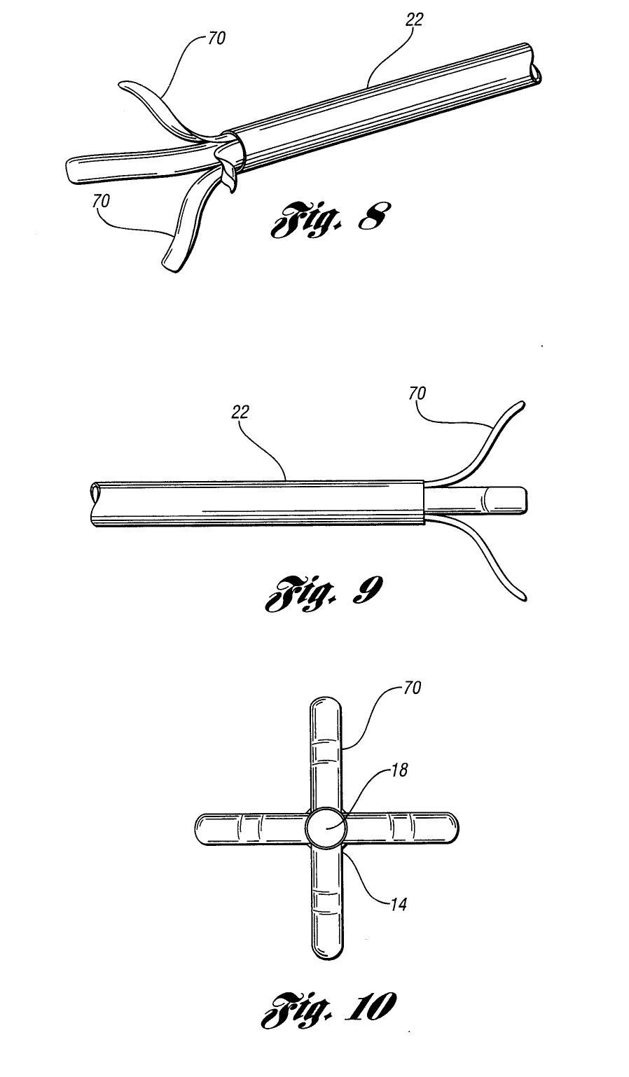

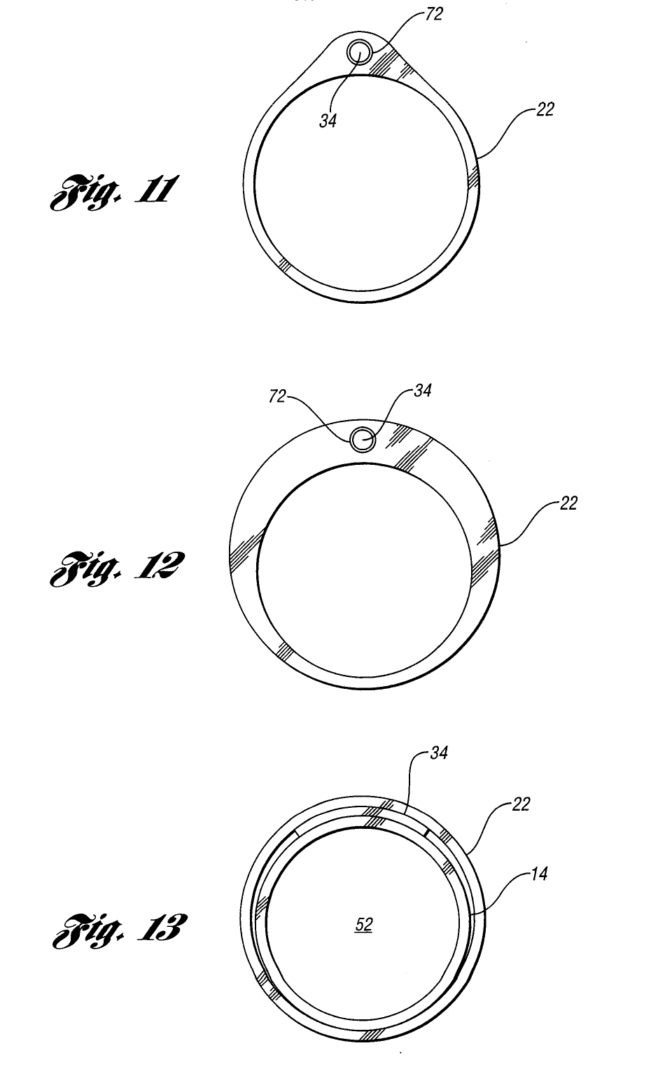

### Other Variations (FIGS. 8-13)

In the sixth embodiment (FIGS. 8-10), the locating member 42 functions similarly to that of embodiment 4, except the struts 70 have a more concave configuration to facilitate their placement against the vessel wall.

Designs depicted in FIGS. 11-13 show optional cross sections of the shaft 20 of the guide catheter 14. Each design shows a circular or flat wire (arcuate ribbon) connecting component 34. FIGS. 11 & 12 depict a tunnel 72 defined within a wall of the sheath 22, through which the connecting components 34 move. These designs could be interchanged in each embodiment (1-6). Interchanging the connecting components could slightly modify the cross sections of each design.

Here are the reference numerals and respective design components:

| Reference No. | Feature |
|---|---|
| 10 | Catheter assembly |
| 12 | ostial lesion |
| 14 | Guide catheter |
| 16 | Proximal end |
| 17 | External devices (of 14) |
| 18 | Distal end |
| 19 | Luer-lock (of 14) |
| 20 | Intermediate section (of 14) |
| 22 | Sheath |
| 24 | Internal lumen (of 22) |
| 26 | Near end (of 22) |
| 28 | Far end (of 22) |
| 30 | Central section (of 22) |
| 32 | Cavity |
| 34 | Connecting component |
| 36 | Proximal end (of 34) |
| 38 | Actuating mechanism |
| 40 | Distal end (of 34) |
| 42 | Ostial locating member |
| 44 | Struts |
| 46 | Feet |
| 48 | Ostial plane |
| 50 | Interior wall of aorta |
| 52 | Inner lumen (of 14) |
| 54 | Ribbon |
| 56 | Distal cage |
| 58 | Basal ring |
| 60 | Apex ring |
| 62 | Links |
| 64 | Open position |
| 66 | Central portion (of 62) |
| 68 | Forward position (of 38) |
| 70 | Legs (of 42) |
| 72 | Tunnel |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed:

1. A catheter system for placing a medical device proximate an ostium of an artery to treat an ostial lesion, the catheter system having
a guide catheter with a proximal end and a distal end;
a sheath that has an internal lumen through which the guide catheter extends, the sheath having a near end and a far end;
a cavity defined between the outside of the guide catheter and the inside of the sheath;
an actuating mechanism associated with the near end of the sheath;
one or more connecting components that extend axially within the cavity, each connecting component having a proximal end that coordinates with the actuating mechanism and a distal end that extends toward the distal end of the guide catheter; and
an ostial locating member that extends from the far end of the sheath so that the ostial locating member lies outside the guide catheter, the ostial locating member having at least two flexible, radially extending struts that terminate with radio-opaque tips that lie in an ostial plane when they are advanced distally against the ostium;
the ostial locating member permitting placement of the radio-opaque tips proximate to an ostial plane when the ostial locating member is advanced distally after the legs have been extended radially outwardly and beyond the distal end of the guide catheter.

2. The catheter system of claim 1 where the diameter of the inner lumen of the guide catheter is between 6 Fr and 8 Fr and the diameter of the outside of the sheath is between 7.5 Fr and 9.5 Fr.

3. The catheter system of claim 1 where the one or more connecting components comprise a ribbon with an arcuate cross section that is accommodated by and can slide axially within the cavity.

4. The catheter system of claim 1, wherein the ostial locating member comprises a cage having a basal ring, an apex ring and flexible, longtitudinal links extending therebetween, so that when the actuating mechanism is in an open position, the one or more connecting components translate axially and the links of the cage expand radially to form a disk-like shape.

5. The catheter system of claim 4, wherein the links have a central portion that includes the radio-opaque material.

6. The catheter system of claim 5, wherein the links when extended radially have an outside diameter that lies between 4 and 15 mm.

7. The catheter system of claim 1, wherein the actuating mechanism is biased to a forward position, so that upon release, the ostial locating member opens.

8. The catheter system of claim 1, wherein the ostial locating member has radially expandable legs.

9. The catheter system of claim 8, wherein the number of expandable legs equals at least four.

10. The catheter system of claim 8 where at least some of the radially expandable legs are curved, so that upon deployment, the ostial locating member assumes a cup-shape.

11. The catheter system of claim 4 where the number of links ≧12.

12. The catheter system of claim 4 wherein the links comprise strands of mesh.

13. The catheter system of claim 12 wherein the mesh is interwoven.

14. The catheter system of claim 4, wherein the links assume a bent configuration when the actuating mechanism moves the one or more connecting components and thus the basal ring of the cage distally, so that the bent configuration lies ahead of the distal end of the guide catheter.

15. The catheter system of claim 1 where the ostial locating member includes Nitinol.

16. The catheter system of claim 1 where the ostial locating member is formed from a shape memory alloy.

17. The catheter system of claim 1 wherein the actuating mechanism includes a knob by which longitudinal or rotational or combined longitudinal and rotational force can be applied to the connecting component by which it can be translated axially.

18. A catheter system for placing a medical device proximate an ostium of an artery to treat an ostial lesion, the catheter system having
a guide catheter with a proximal end and a distal end;
a sheath having a wall that has an internal lumen through which the guide catheter extends, the sheath having a near end and a far end;
an actuating mechanism associated with the near end of the sheath;
an axially extending tunnel defined within the wall of the sheath;
one or more connecting components that extend axially through the tunnel, each connecting component having a proximal end that coordinates with the actuating mechanism and a distal end that extends toward the distal end of the guide catheter; and
an ostial locating member that extends from the far end of the sheath so that the ostial locating member lies outside the guide catheter, the ostial locating member having at least two flexible, radially extending struts that terminate with radio-opaque tips that lie in an ostial plane when they are advanced distally against the ostium;
the ostial locating member permitting placement of the radio-opaque tips proximate to or in an ostial plane when the ostial locating member is advanced distally after the legs have been extended radially outwardly and beyond the distal end of the guide catheter.

19. A method for placing a medical device proximate an ostium of an artery to treat an ostial lesion, comprising the steps of:
providing a catheter system having a guide catheter and a sheath with an internal lumen through which the guide catheter extends;
deploying an activating mechanism that is associated with the near end of the sheath so that one or more connecting components that extend axially between the outside of the catheter and the inside of the sheath are translated longitudinally, thereby deploying by movement of the actuating mechanism an ostial locating member that lies ahead of the distal end of the guide catheter; and
advancing the ostial locating member toward the ostium so that legs associated with the ostial locating member lie in or near the ostial plane.
